SEQUENCE LISTING (1 sequence)

SEQ ID NO: 1 - DNA, 147 bp, Homo sapiens
ctatagggcggccgggaattcgtcgactggatccggtaccgaggagatctgccgccgcgatcgccggcgcgccagatctcaagcttaactagttagcggaccgacgcgttaagcggccgcactcgaggtttaaacggccggccgcgg

(19) United States
(12) Patent Application Publication
(10) Pub. No.: US 2015/0353623 A1
(43) Pub. Date: Dec. 10, 2015

(54) SUBSTANCES AND METHODS FOR THE TREATMENT OF CEREBRAL AMYLOID ANGIOPATHY RELATED CONDITIONS OR DISEASES

(71) Applicants: Loma Linda University, Loma Linda, CA (US); Loma Linda University Medical Center, Loma Linda, CA (US); University of California, Los Angeles, Los Angeles, CA (US); University of California, Irvine, Irvine, CA (US); North Carolina State University, Raleigh, CA (US)

(72) Inventors: Wolff M. Kirsch, Redlands, CA (US); Andrew Crofton, Loma Linda, CA (US); Tanya Cupino, Loma Linda, CA (US); Charles Glabe, Irvine, CA (US); Samuel M. Hudson, Raleigh, NC (US); Matthew Schrag, New Haven, CT (US); Harry V. Vinters, Los Angeles, CA (US); Matthew Zabel, Bethesda, MD (US)

(21) Appl. No.: 14/677,953

(22) Filed: Apr. 2, 2015

Related U.S. Application Data
(60) Provisional application No. 61/974,901, filed on Apr. 3, 2014.

(51) Int. Cl. C07K 14/705; A61K 31/713; A61K 45/06; A61K 31/192; C07K 16/18

(52) U.S. Cl. CPC C07K 14/70596; A61K 31/192; C07K 16/18; A61K 45/06; A61K 31/713

(57) ABSTRACT

A substance for treating a cerebral amyloid angiopathy related condition or disease affecting cerebrovasculature in a patient, comprising an inhibitor that causes inhibition of the formation of membrane attack complex of the complement system; and a vehicle for transporting the inhibitor into the cerebrovasculature; where the inhibition by the inhibitor is sufficient to decrease the incidence of or to prevent the incidence of cytolysis of the smooth muscle cells. A method for treating a cerebral amyloid angiopathy related condition or disease affecting cerebrovasculature in a patient, comprising: a) identifying a patient with a cerebral amyloid angiopathy related condition or disease; b) providing one or more than one substance that comprises an inhibitor that causes inhibition of the formation of membrane attack complex of the complement system, c) administering one or more than one dose of the one or more than one substance to the patient.

### INCORPORATION BY REFERENCE TO RELATED APPLICATIONS

Any and all priority claims identified in the Application Data Sheet, or any correction thereto, are hereby incorporated by reference under 37 CFR 1.57. This application claims priority to U.S. Provisional Application No. 61/974,901, filed Apr. 3, 2014. The aforementioned application is incorporated by reference herein in its entirety, and is hereby expressly made a part of this specification.

### STATEMENT REGARDING FEDERALLY SPONSORED RESEARCH OR DEVELOPMENT

This invention was made with United States Government support under Grant No. 5 RO1 AG020948, awarded by the National Institutes of Health/National Institute on Aging. The United States Government has certain rights in this invention.

### BACKGROUND

Cerebral amyloid angiopathy (CAA) is a nonspecific disease entity that has been associated with a number of neuropathological conditions, including a 70-90% prevalence in patients diagnosed with Alzheimer's disease (AD). Cerebral amyloid angiopathy is characterized by the pathologic accumulation of beta-amyloid (amyloid β, Aβ) plaques in the tunica media and tunica adventitia of small and mid-sized arteries (and less frequently of veins) of the cerebral cortex and the leptomeninges resulting in vascular fragility, intracranial bleeding (lobar intracerebral hemorrhage) and in some cases dementia.

Currently, there is no known effective treatment to decrease or prevent the underlying deposition of beta-amyloid that characterizes cerebral amyloid angiopathy. Though beta-amyloid immunotherapy rapidly clears amyloid plaques, cerebral amyloid angiopathy is worsened with increased brain inflammation and increased brain microbleeds. Therefore, the current goal of treatment is symptomatic, and includes physical rehabilitation and amelioration of seizures when present.

### SUMMARY

There is a need for a method for treating cerebral amyloid angiopathy.

According to one embodiment, there is provided a substance for treating a cerebral amyloid angiopathy related condition or disease affecting cerebrovasculature in a patient, where the cerebrovasculature comprises tunica intima comprising endothelial cells, tunica media comprising smooth muscle cells, and tunica adventitia, where the condition or disease is associated with an incidence of cytolysis of the smooth muscle cells from beta-amyloid deposition in the cerebrovasculature that leads to formation of membrane attack complex of the complement system in the smooth muscle cells. The substance comprises one or more than one inhibitor that causes inhibition of the formation of membrane attack complex of the complement system, and one or more than one vehicle for transporting the one or more than one inhibitor into the cerebrovasculature, where the inhibition by the inhibitor is sufficient to decrease the incidence of or to prevent the incidence of cytolysis of the smooth muscle cells. In one embodiment, the substance crosses the tunica intima and enters the tunica media. In one embodiment, the patient has a blood brain barrier comprising the tunica intima, the tunica media and the tunica adventitia, and where the substance crosses the blood brain barrier. In one embodiment, the one or more than one inhibitor is a plurality of inhibitors. In another embodiment, the plurality of inhibitors is two inhibitors. In another embodiment, the plurality of inhibitors is three inhibitors. In another embodiment, the plurality of inhibitors is four inhibitors. In one embodiment, the cerebral amyloid angiopathy related condition or disease is selected from the group consisting of one or more than one of Alzheimer's disease, a brain microbleed, cerebral amyloidosis of the parenchyma, mild cognitive impairment with amyloid plaques in the brain and a combination of the preceding. In one embodiment, the one or more than one inhibitor specifically causes inhibition of the formation of membrane attack complex of the complement system in the tunica intima of the cerebrovasculature, tunica media of the cerebrovasculature, or both the tunica intima of the cerebrovasculature and tunica media of the cerebrovasculature. In one embodiment, one or more than one of the one or more than one inhibitor that causes inhibition of the formation of membrane attack complex of the complement system upregulates CD59 glycoprotein levels in the cerebrovasculature of the patient. In one embodiment, one or more than one of the one or more than one inhibitor that causes inhibition of the formation of membrane attack complex of the complement system is selected from the group consisting of one or more than one of alphaGal lectin, anti-C5 Mab, C1-Inhibitor, factor H, human CD59 cDNA, and a combination of the preceding. In one embodiment, one of the one or more than one inhibitor that causes inhibition of the formation of membrane attack complex of the complement system is a plasmid comprising human CD59 cDNA. In one embodiment, one of the one or more than one inhibitor comprises human CD59 cDNA in the pCMV6-AC plasmid, or human CD59 cDNA in the pCMV6-XL5 plasmid. In one embodiment, the one or more than one vehicle is a plurality of vehicles. In another embodiment, the plurality of vehicles is two vehicles. In another embodiment, the plurality of vehicles is three vehicles. In another embodiment, the plurality of vehicles is four vehicles. In one embodiment, one or more than one of the one or more than one vehicle is selected from the group consisting of chitosan nanoparticles, colloidal metallic nanoparticles, polymer nanoparticles and viral particles. In one embodiment, at least one of the one or more than one vehicle comprises chitosan nanoparticles. In one embodiment, the substance comprises one vehicle and a plurality of inhibitors. In another embodiment, the substance comprises one vehicle and two inhibitors. In one embodiment, the substance further comprises one or more than one targeting agent that recognizes the beta-amyloid deposited in the cerebrovasculature, where one or more than one targeting agent forms at least part of the surface of the substance when one or more than one targeting agent is combined with the inhibitor and the vehicle, thereby directing the substance to the beta-amyloid deposited in the cerebrovasculature when the substance is administered to the patient. In one embodiment, the targeting agent recognizes and attaches to a subset of conformationally unique beta-amyloid deposited in the cerebrovasculature, where the conformationally unique beta-amyloid deposited in the cerebrovasculature is specific for cerebral amyloid angiopathy. In one embodiment, when combined with the inhibitor and the vehicle, the targeting agent directs the substance to the beta-amyloid deposited in the cerebrovascular smooth muscle cells when the substance is administered to the patient. In one embodiment, at least one of the one or more than one targeting agent is a monoclonal antibody or is a fragment of a monoclonal antibody. In one embodiment, at least one of the one or more than one targeting agent is selected from the group consisting of amyloid antibody (M31) Fab fragments, 6E1O beta-amyloid monoclonal antibody and a combination of the preceding. In one embodiment, the one or more than one targeting agent is a plurality of targeting agents. In another embodiment, the plurality of targeting agents is two targeting agents. In another embodiment, the plurality of targeting agents is three targeting agents. In another embodiment, the plurality of targeting agents is four targeting agents. In one embodiment, the substance further comprises one or more than one additional chemical that enables determination of plasmid transfection efficacy where the inhibitor is a plasmid, or enables tracking of the substance. In one embodiment, at least one of the one or more than one additional chemical is selected from the group consisting of hydroxycoumarin and green fluorescent protein.

According to another embodiment, there is provided a pharmaceutical for treating a cerebral amyloid angiopathy related condition or disease. The pharmaceutical comprises one or more than one substance according to the embodiments, and further comprises one or more than one of a binder, a buffer, a coloring chemical, a flavoring chemical and a preservative.

According to another embodiment, there is provided a method for treating a cerebral amyloid angiopathy related condition or disease affecting cerebrovasculature in a patient, where the cerebrovasculature comprises tunica intima comprising endothelial cells, tunica media comprising smooth muscle cells, and tunica adventitia, where the condition or disease is associated with an incidence of cytolysis of the smooth muscle cells from beta-amyloid deposition in the cerebrovasculature that leads to formation of membrane attack complex of the complement system in the smooth muscle cells. The method comprises a) identifying a patient with a cerebral amyloid angiopathy related condition or disease suitable for treatment; b) providing one or more than one substance that comprises an inhibitor that causes inhibition of the formation of membrane attack complex of the complement system, or comprises providing one or more than one pharmaceutical comprising one or more than one substance that comprises an inhibitor that causes inhibition of the formation of membrane attack complex of the complement system, or comprises providing both one or more than one substance that comprises an inhibitor that causes inhibition of the formation of membrane attack complex of the complement system and one or more than one pharmaceutical comprising one or more than one substance that comprises an inhibitor that causes inhibition of the formation of membrane attack complex of the complement system, where the inhibition by the inhibitor is sufficient to decrease the incidence of or to prevent the incidence of cytolysis of the smooth muscle cells; and c) administering one or more than one dose of the one or more than one substance or administering one or more than one dose of the one or more than one pharmaceutical to the patient by a route. In one embodiment, the patient is a human. In another embodiment, the cerebral amyloid angiopathy related condition or disease is selected from the group consisting of one or more than one of Alzheimer's disease, a brain microbleed, cerebral amyloidosis of the parenchyma, mild cognitive impairment with amyloid plaques in the brain and a combination of the preceding. In one embodiment, identifying the patient comprises consulting patient records to determine if the patient has a cerebral amyloid angiopathy related condition or disease suitable for treatment. In one embodiment, identifying the patient comprises diagnosing the patient with a cerebral amyloid angiopathy related condition or disease suitable for treatment. In a preferred embodiment, diagnosing the patient comprises performing one or more than one of action selected from the group consisting of identifying one or more than one marker for cerebral amyloid angiopathy in blood or another body fluid of the patient, performing cognitive testing, performing an invasive procedure, performing a non-invasive imaging procedure, and performing a physical examination. In one embodiment, one or more than one of the one or more than one substance is a substance according to the embodiments. In one embodiment, one or more than one of the one or more than one pharmaceutical is a pharmaceutical according to the embodiments. In one embodiment, the one or more than one substance is a plurality of substances. In another embodiment, the one or more than one substance is two substances. In one embodiment, the one or more than one pharmaceutical is a plurality of pharmaceuticals. In another embodiment, the one or more than one pharmaceutical is two pharmaceuticals. In one embodiment, the one or more than one dose is one dose. In another embodiment, the one or more than one dose is a plurality of doses. In another embodiment, the plurality of doses is two doses. In another embodiment, the plurality of doses is three doses. In another embodiment, the plurality of doses is four doses. In another embodiment, the plurality of doses is more than four doses. In one embodiment, the one or more than one dose is administered daily for a predetermined amount of time. In another embodiment, the one or more than one dose is administered twice daily for a predetermined amount of time. In another embodiment, the one or more than one dose is administered weekly for a predetermined amount of time. In another embodiment, the one or more than one dose is administered monthly for a predetermined amount of time. In another embodiment, the one or more than one dose is administered between once a day and once a week for a predetermined amount of time. In another embodiment, the one or more than one dose is administered between once a day and once a month for a predetermined amount of time. In another embodiment, the dose is between 0.000001 mg/m² body surface area and 100 g/m² body surface area. In another embodiment, the dose is between 0.0001 mg/m² body surface area and 10 g/m² body surface area. In another embodiment, the dose is between 1 mg/m² body surface area and 1 g/m² body surface area. In one embodiment, the route is selected from the group consisting of intra-arterial injection, intramuscular injection, intranasal spray and intravenous injection. In one embodiment, the method further comprises determining the effect of treatment on the patient. In one embodiment, determining the effect of treatment on the patient comprises performing one or more than one of action selected from the group consisting of identifying one or more than one marker for cerebral amyloid angiopathy in blood or another body fluid of the patient, performing cognitive testing, performing an invasive procedure, performing a non-invasive imaging procedure, and performing a physical examination. In one embodiment, the method further comprises adjusting treatment. In another embodiment, adjusting treatment comprises administering to the patient one or more than one additional dose of the one or more than one substance or administering one or more than one additional dose of the one or more than one pharmaceutical to the patient. In one embodiment, the substance further comprises one or more than one vehicle for transporting the one or more than one inhibitor into the cerebrovasculature. In one embodiment, the substance crosses the tunica intima and enters the tunica media. In one embodiment, the patient has a blood brain barrier comprising the tunica intima, the tunica media and the tunica adventitia, and where the substance crosses the blood brain barrier. In one embodiment, the one or more than one inhibitor is a plurality of inhibitors. In another embodiment, the plurality of inhibitors is two inhibitors. In another embodiment, the plurality of inhibitors is three inhibitors. In another embodiment, the plurality of inhibitors is four inhibitors. In one embodiment, the one or more than one inhibitor specifically causes inhibition of the formation of membrane attack complex of the complement system in the tunica intima of the cerebrovasculature, tunica media of the cerebrovasculature, or both the tunica intima of the cerebrovasculature and tunica media of the cerebrovasculature. In one embodiment, one or more than one of the one or more than one inhibitor that causes inhibition of the formation of membrane attack complex of the complement system upregulates CD59 glycoprotein levels in the cerebrovasculature of the patient. In one embodiment, one or more than one of the one or more than one inhibitor that causes inhibition of the formation of membrane attack complex of the complement system is selected from the group consisting of one or more than one of alphaGal lectin, anti-C5 Mab, C1-Inhibitor, factor H, human CD59 cDNA, and a combination of the preceding. In one embodiment, one of the one or more than one inhibitor that causes inhibition of the formation of membrane attack complex of the complement system is a plasmid comprising human CD59 cDNA. In one embodiment, one of the one or more than one inhibitor comprises human CD59 cDNA in the pCMV6-AC plasmid, or human CD59 cDNA in the pCMV6-XL5 plasmid. In one embodiment, the one or more than one vehicle is a plurality of vehicles. In another embodiment, the plurality of vehicles is two vehicles. In another embodiment, the plurality of vehicles is three vehicles. In another embodiment, the plurality of vehicles is four vehicles. In one embodiment, the one or more than one vehicle crosses the tunica intima and enters the tunica media. In another embodiment, the patient has a blood brain barrier comprising the tunica intima, the tunica media and the tunica adventitia, and the one or more than one vehicle crosses the blood brain barrier. In one embodiment, one or more than one of the one or more than one vehicle is selected from the group consisting of chitosan nanoparticles, colloidal metallic nanoparticles, polymer nanoparticles and viral particles. In another embodiment, at least one of the one or more than one vehicle comprises chitosan nanoparticles. In another embodiment, the substance comprises one vehicle and a plurality of inhibitors. In another embodiment, the substance comprises one vehicle and two inhibitors. In another embodiment, the method further comprises one or more than one targeting agent that recognizes the beta-amyloid deposited in the cerebrovasculature, where one or more than one targeting agent forms at least part of the surface of the substance when one or more than one targeting agent is combined with the inhibitor and the vehicle, thereby directing the substance to the beta-amyloid deposited in the cerebrovasculature when the substance is administered to the patient. In one embodiment, the targeting agent recognizes and attaches to a subset of conformationally unique beta-amyloid deposited in the cerebrovasculature, where the conformationally unique beta-amyloid deposited in the cerebrovasculature is specific for cerebral amyloid angiopathy. In one embodiment, when combined with the inhibitor and the vehicle, the targeting agent directs the substance to the beta-amyloid deposited in the cerebrovascular smooth muscle cells when the substance is administered to the patient. In one embodiment, at least one of the one or more than one targeting agent is a monoclonal antibody or is a fragment of a monoclonal antibody. In one embodiment, at least one of the one or more than one targeting agent is selected from the group consisting of amyloid antibody (M31) Fab fragments, 6E10 beta-amyloid monoclonal antibody and a combination of the preceding. In one embodiment, the one or more than one targeting agent is a plurality of targeting agents. In another embodiment, the plurality of targeting agents is two targeting agents. In another embodiment, the plurality of targeting agents is three targeting agents. In another embodiment, the plurality of targeting agents is four targeting agents. In one embodiment, the substance further comprises one or more than one additional chemical that enables determination of plasmid transfection efficacy where the inhibitor is a plasmid, or enables tracking of the substance. In one embodiment, at least one of the one or more than one additional chemical is selected from the group consisting of hydroxycoumarin and green fluorescent protein. In one embodiment, the pharmaceutical further comprises one or more than one of a binder, a buffer, a coloring chemical, a flavoring chemical and a preservative.

### BRIEF DESCRIPTION OF THE DRAWINGS

These and other features, aspects and advantages of the present invention will become better understood with regard to the following description, appended claims, and accompanying drawings where:

FIG. 1 is a schematic depiction of one embodiment of the human CD59 cDNA in the pCMV6-AC;

FIG. 2 shows western blots showing the accumulation of C6 and beta-actin (a protein expressed in all cells and used as a loading control to normalize C6 values) in blood vessel walls from control brain tissue (left), in blood vessel walls from brain tissue from patients diagnosed with Alzheimer's disease (AD, center), and in blood vessel walls from brain tissue from patients diagnosed with Alzheimer's disease concurrent with cerebral amyloid angiopathy (AD/CAA, right);

FIG. 3 is a histogram showing the average concentration of C6 from multiple western blots (n=4) in blood vessel walls from control brain tissue, in blood vessel walls from brain tissue from patients diagnosed with Alzheimer's disease (AD), and in blood vessel walls from brain tissue from patients diagnosed with Alzheimer's disease concurrent with cerebral amyloid angiopathy (AD/CAA);

FIG. 4 are photomicrographs showing C6 localization in the walls of blood vessels from control brain tissue (left), in the walls of blood vessels from brain tissue from patients diagnosed with Alzheimer's disease (AD, center), and in the walls of blood vessels from brain tissue from patients diagnosed with Alzheimer's disease concurrent with cerebral amyloid angiopathy (AD/CAA, right) by immunohisto-chemistry with DAB-staining;

FIG. 5 are confocal micrographs of microglia walls from control brain tissue (A, left), blood vessel walls from brain tissue from patients diagnosed with Alzheimer's disease (B, center), and blood vessel walls from brain tissue from patients diagnosed with Alzheimer's disease concurrent with cerebral amyloid angiopathy (C, right), where the brain tissue was stained for alpha2-macroglobulin and beta-amyloid (A, B), and for beta-amyloid and CD11b (C); and

FIG. 6 illustrates nanoparticle impact on cell viability. Y-axis is absorbance at 495 nm, in arbitrary units.

### DETAILED DESCRIPTION

According to one embodiment, there is provided a substance for treating a cerebral amyloid angiopathy related condition or disease affecting cerebrovasculature in a patient, where the cerebrovasculature comprises tunica intima comprising endothelial cells, tunica media comprising smooth muscle cells, and tunica adventitia, where the condition or disease is associated with an incidence of cytolysis of the smooth muscle cells from beta-amyloid deposition in the cerebrovasculature that leads to formation of membrane attack complex of the complement system in the smooth muscle cells, where the substance comprises one or more than one inhibitor that causes inhibition of the formation of membrane attack complex of the complement system, and where the one or more than one inhibition by the inhibitor is sufficient to decrease the incidence of or to prevent the incidence of cytolysis of the smooth muscle cells. In one embodiment, the substance further comprises a vehicle and a targeting agent. According to another embodiment, there is provided a pharmaceutical for treating a cerebral amyloid angiopathy related condition or disease. The pharmaceutical comprises a substance according to the embodiments. According to another embodiment, there is provided a method for treating a cerebral amyloid angiopathy related condition or disease affecting cerebrovasculature in a patient, where the cerebrovasculature comprises tunica intima comprising endothelial cells, tunica media comprising smooth muscle cells, and tunica adventitia, where the condition or disease is associated with an incidence of cytolysis of the smooth muscle cells from beta-amyloid deposition in the cerebrovasculature that leads to formation of membrane attack complex of the complement system in the smooth muscle cells. The method comprises providing one or more than one substance that comprises an inhibitor that causes inhibition of the formation of membrane attack complex of the complement system, or comprises providing one or more than one pharmaceutical comprising one or more than one substance that comprises an inhibitor that causes inhibition of the formation of membrane attack complex of the complement system, or comprises providing both one or more than one substance that comprises an inhibitor that causes inhibition of the formation of membrane attack complex of the complement system and one or more than one pharmaceutical comprising one or more than one substance that comprises an inhibitor that causes inhibition of the formation of membrane attack complex of the complement system, where the inhibition by the inhibitor is sufficient to decrease the incidence of or to prevent the incidence of cytolysis of the smooth muscle cells, and administering one or more than one dose of one or more than one substance or of the one or more than one pharmaceutical to the patient by a route. In one embodiment, one or more than one of the one or more than one substance is a substance according to the embodiments. In another embodiment, one or more than one of the one or more than one pharmaceutical is a pharmaceutical according to the embodiments. In one embodiment, the cerebral amyloid angiopathy related condition or disease is selected from the group consisting of one or more than one of Alzheimer's disease, a brain microbleed, cerebral amyloidosis of the parenchyma, mild cognitive impairment with amyloid plaques in the brain and a combination of the preceding. The substances, methods and pharmaceuticals will now be disclosed in detail.

As used in this disclosure, except where the context requires otherwise, the term “comprise” and variations of the term, such as “comprising,” “comprises” and “comprised” are not intended to exclude other additives, components, integers or steps.

As used in this disclosure, except where the context requires otherwise, the method steps disclosed are not intended to be limiting nor are they intended to indicate that each step is essential to the method or that each step must occur in the order disclosed.

As used herein, except where the context requires otherwise, the term “cerebrovasculature” and related terms refer to both cerebral vasculature and leptomeningeal vasculature.

As used in this disclosure, except where the context requires otherwise, “cerebral amyloid angiopathy related condition or disease” means “cerebral amyloid angiopathy” and any condition or disease that includes “cerebral amyloid angiopathy” as a component of the condition or disease, such as for example Alzheimer's disease, a brain microbleed, cerebral amyloidosis of the parenchyma, mild cognitive impairment with amyloid plaques in the brain. Further, “cerebral amyloid angiopathy related condition or disease” means “one or more than one cerebral amyloid angiopathy related condition, one or more than one cerebral amyloid angiopathy related disease, or both one or more than one cerebral amyloid angiopathy related condition and one or more than one cerebral amyloid angiopathy related disease.”

According to one embodiment, there is provided a substance for treating a cerebral amyloid angiopathy related condition or disease affecting cerebrovasculature in a patient, where the cerebrovasculature comprises tunica intima comprising endothelial cells, tunica media comprising smooth muscle cells, and tunica adventitia, where the condition or disease is associated with an incidence of cytolysis of the smooth muscle cells from beta-amyloid deposition in the cerebrovasculature that leads to formation of membrane attack complex of the complement system in the smooth muscle cells, where the substance comprises one or more than one inhibitor that causes inhibition of the formation of membrane attack complex of the complement system, and where the inhibition by the one or more than one inhibitor is sufficient to decrease the incidence of or to prevent the incidence of cytolysis of the smooth muscle cells. In one embodiment, the substance crosses the tunica intima and enters the tunica media. In another embodiment, the patient has a blood brain barrier comprising the tunica intima, the tunica media and the tunica adventitia, and the substance crosses the blood brain barrier.

In one embodiment, the one or more than one inhibitor is a plurality of inhibitors, such as for example both alphaGal lectin and human CD59 cDNA. In another embodiment, the plurality of inhibitors is two inhibitors. In another embodiment, the plurality of inhibitors is three inhibitors. In another embodiment, the plurality of inhibitors is four inhibitors. In one embodiment, the cerebral amyloid angiopathy related condition or disease is selected from the group consisting of one or more than one of Alzheimer's disease, a brain microbleed, cerebral amyloidosis of the parenchyma, mild cognitive impairment with amyloid plaques in the brain and a combination of the preceding. In a preferred embodiment, the one or more than one inhibitor specifically causes inhibition of the formation of membrane attack complex of the complement system in the tunica intima of the cerebrovasculature, tunica media of the cerebrovasculature, or both the tunica intima of the cerebrovasculature and tunica media of the cerebrovasculature, where specific inhibition of membrane attack complex is determined by a decrease of cell lysis after applying a predetermined amount of activated complement and the inhibitor as compared to amount of cell lysis with the same amount of activated complement without the inhibition. As will be understood by those with skill in the art with respect to this disclosure, specific inhibition can be measured either directly by increased light absorbance when the cells tested are erythrocytes or indirectly by increased light absorbance in proportion to overall metabolic activity in an MTT assay, or by any other suitable method according to techniques known to those with skill in the art.

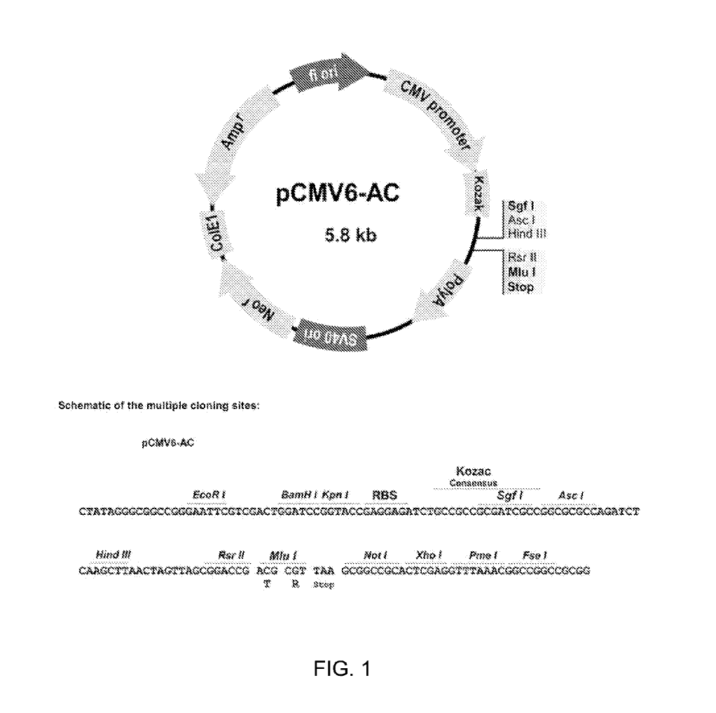

In one embodiment, one or more than one of the one or more than one inhibitor that causes inhibition of the formation of membrane attack complex of the complement system upregulates CD59 glycoprotein levels in the cerebrovasculature of the patient. In one embodiment, one or more than one of the one or more than one inhibitor that causes inhibition of the formation of membrane attack complex of the complement system is selected from the group consisting of one or more than one of alphaGal lectin (a lectin that binds to terminal Gal-alpha(1-3)Gal residues) which increases CD59 glycoprotein levels by induced ligation), anti-C5 Mab (binds component 5 of the complement system thereby blocking component 5 from potentiating downstream complement molecules), C1-Inhibitor (prevents initiation of the complement cascade), factor H (accelerates decay of the C3 convertase), human CD59 cDNA (interrupts formation of the transmembrane pore in membrane attack complex), and a combination of the preceding. In a preferred embodiment, one of the one or more than one inhibitor that causes inhibition of the formation of membrane attack complex of the complement system is a plasmid comprising human CD59 cDNA. In a particularly preferred embodiment, one of the one or more than one inhibitor comprises human CD59 cDNA in the pCMV6-AC. Referring now to FIG. 1, there is shown a schematic depiction of one embodiment of the human CD59 cDNA in the pCMV6-AC that can be used as an inhibitor according to the embodiments. As can be seen, without the CD59 cDNA, the pCMV6-AC plasmid is 5.8 kb. With the CD59 open reading frame (OriGene RC218343) the entire construct is 6.1 kb. The pCMV6-AC has the following key features for mammalian expression: cytomegalovirus (CMV) promoter (strong, constitutive, ubiquitous expression), and human growth hormone polyA (hGH polyA) signal sequence (leads to polyadenylation of the transcribed mRNA which is essential for mRNA stability in cells). The pCMV6-AC also comprises a modified bacterial origin of replication (allows for enhanced replication in E. coli to produce more plasmids), and a Neomycin phosphotransferase locus (confers Neomycin resistance and allows for selection of plasmid-containing bacteria or mammalian cells). As will be understood by those with skill in the art with respect to this disclosure, though the plasmid disclosed above and shown in FIG. 1 is suitable for the inhibitor according to the embodiments, other plasmids comprising human CD59 gene that function as intended in the substance can also be used, such as for example the pCMV6-XL5 entry vector which is 4.7 kb that is identical to pCMV6-AC except that it lacks the Neomycin phosphotransferase locus and includes a T7 promoter for cell-free in vitro replication systems. As will be understood by those with skill in the art with respect to this disclosure, there are several commercially suitable splice variants of CD59 in plasmids suitable for use in the embodiments.

In one embodiment, the substance for treating a cerebral amyloid angiopathy related condition or disease affecting cerebrovasculature in a patient further comprises one or more than one vehicle for transporting the one or more than one inhibitor that causes inhibition of the formation of membrane attack complex of the complement system into the cerebrovasculature. In one embodiment, one or more than one of the one or more than one vehicle is selected from the group consisting of chitosan nanoparticles, colloidal metallic nanoparticles, polymer nanoparticles and viral particles. In one embodiment, the one or more than one vehicle is a plurality of vehicles. In a preferred embodiment, the plurality of vehicles are chitosan nanoparticles having a maximum diameter of between 50-100 nm, and chitosan nanoparticles having a maximum diameter of between 200-400 nm. In a preferred embodiment, the plurality of vehicles are chitosan nanoparticles having a maximum diameter of between 50-100 nm, and chitosan nanoparticles having a maximum diameter of between 400-600 nm. In a preferred embodiment, the plurality of vehicles are chitosan nanoparticles having a maximum diameter of between 200-400 nm, and chitosan nanoparticles having a maximum diameter of between 400-600 nm. In a preferred embodiment, the plurality of vehicles are chitosan nanoparticles having a maximum diameter of between 50-100 nm, chitosan nanoparticles having a maximum diameter of between 200-400 nm, and chitosan nanoparticles having a maximum diameter of between 400-600 nm. In another embodiment, the plurality of vehicles is two vehicles. In another embodiment, the plurality of vehicles is three vehicles. In another embodiment, the plurality of vehicles is four vehicles. In a preferred embodiment, the one or more than one vehicle crosses the tunica intima and enters the tunica media. In another embodiment, the patient has a blood brain barrier comprising the tunica intima, the tunica media and the tunica adventitia, and the one or more than one vehicle crosses the blood brain barrier. In a preferred embodiment, at least one of the one or more than one vehicle comprises chitosan nanoparticles. Chitosan is a deacetylated product of chitin, a polysaccharide found in the internal structures and outer skeleton of some invertebrates including crabs, insects, lobsters and shrimps. Chitin is composed of β(1-4) linked units of the amino sugar N-acetyl-glucosamine linked, and is the main source for the production of chitosan. Medical grade chitosan (low protein, medium-high molecular weight) suitable for use in the embodiments can be obtained from Scion Cardio-Vascular, Inc. Miami, Fla. US, though any suitable source can be used, as will be understood by those with skill in the art with respect to this disclosure. Chitosan nanoparticles are particularly suited as a vehicle according to the embodiments as chitosan is a biologically well-tolerated and biodegradable, and the molecular properties of chitosan allow for easy encapsulation of the inhibitor by chitosan by readily available encapsulation techniques. Further chitosan nanoparticles have positive surface charges that allow for easy surface incorporation of the targeting agent for delivery of the substance to the cerebrovasculature, such as for example by using biotin/avidin conjugation methods, as will be understood by those with skill in the art with respect to this disclosure. Further, chitosan nanoparticles pass through the blood brain barrier due to the positive charges on the chitosan surface, thereby serving as an appropriate vehicle for delivery of the inhibitor to the cerebrovasculature according to the embodiments. Additionally, chitosan nanoparticles without targeting agents are eliminated from the systemic circulation on the basis of particle size by either the kidney glomerular basement membrane for small nanoparticles or by degradation of larger nanoparticles in the liver and reticulo-endothelial system. Further, since chitosan has a structural similarity to sugars, chitosan acts as a cryo-protectant for proteins conjugated to the surface during lyophilization. Additionally, chitosan as the vehicle improves transfection efficiency when the inhibitor comprises a gene, such as for example transfection efficiency when the inhibitor is human CD59 cDNA. As will be understood by those with skill in the art with respect to this disclosure, though chitosan nanoparticles disclosed above are suitable for the vehicle according to the embodiments, other vehicles that function as intended in the substance can also be used.

In one embodiment, the substance comprises one vehicle and a plurality of inhibitors, such as for example a substance comprising chitosan nanoparticles having a maximum diameter of between 200-400 nm as the vehicle and two or more than two inhibitors selected from the group consisting of alphaGal lectin, anti-C5 Mab, C1-Inhibitor, factor H and human CD59 cDNA within the chitosan nanoparticles. In one embodiment, the substance comprises one vehicle and two inhibitors, such as for example a substance comprising chitosan nanoparticles having a maximum diameter of between 200-400 nm as the vehicle and alphaGal lectin and human CD59 cDNA as the inhibitors within the chitosan nanoparticles.

Chitosan nanoparticles as a vehicle according to the embodiments can be made by any suitable method, as will be understood by those with skill in the art with respect to this disclosure. By way of example, in one embodiment, the vehicle is chitosan nanoparticles and the substance is produced as follows. Chitosan of a known molecular weight is dissolved (0.1% w/v) in 4.6 mM HCL and syringe filtered through 0.22 um to remove undissolved residues. After filtration, the pH of the chitosan solution is adjusted to pH 5 with 1 N NaOH. Tripolyphosphate (TPP) solution is prepared in ultra-pure water and the pH corrected to pH 5. pCMV6 plasmids containing the human CD59 cDNA, alphaGal lectin, or any other inhibitor according to the embodiments is added to the TPP solution at an appropriate concentration prior to mixing with the chitosan solution. While under magnetic stirring at 300 rpm, 2 ml of TPP solution is added to 14 ml of the chitosan solution. Nucleation of chitosan particles is spontaneous under these conditions. Stirring is continued for 45 minutes, after which the reaction is left undisturbed for 16 hours at room temperature. The resultant chitosan nanoparticles are 1000 nm (1 micron) or less in maximum diameter. The chitosan nanoparticles formed were 85% deacetylated and depyrogenated.

In one embodiment, the substance further comprises one or more than one targeting agent that recognizes the beta-amyloid deposited in the cerebrovasculature, where one or more than one targeting agent forms at least part of the surface of the substance when one or more than one targeting agent is combined with the inhibitor and the vehicle, thereby directing the substance to the beta-amyloid deposited in the cerebrovasculature when the substance is administered to the patient. In one embodiment, the targeting agent recognizes and attaches to a subset of conformationally unique beta-amyloid deposited between the cerebrovasculature, where the conformationally unique beta-amyloid deposited in the cerebrovasculature is specific for cerebral amyloid angiopathy. In another embodiment, when combined with the inhibitor and the vehicle, the targeting agent directs the substance to the beta-amyloid deposited in the cerebrovascular smooth muscle cells when the substance is administered to the patient. In one embodiment, at least one of the one or more than one targeting agent is a monoclonal antibody or is a fragment of a monoclonal antibody. In one embodiment, the targeting agent is selected from the group consisting of amyloid antibody (M31) Fab fragments (M31 monoclonal antibody is also known as rat anti M311HP1; heterochromatin protein 1 homolog beta) (catalog number MBS214105, MyBioSource, Inc., San Diego, Calif., US), 6E1O beta amyloid monoclonal antibody (also known as Alzheimer disease amyloid protein; amyloid beta A protein; beta-amyloid peptide; cerebral vascular amyloid peptide peptidase; nexin-II; preA4; and protease nexin-II) (catalog number SIG-39300, Covance, Inc., San Diego, Calif., US), and a combination of the preceding, however, any targeting agent suitable for the purpose intended can be used as will be understood by those with skill in the art with respect to this disclosure. Amyloid antibody (M31) Fab fragments are rabbit monoclonal IgG specific for the subset of human and murine vascular amyloid beta assemblies that characterize cerebral amyloid angiopathy. In one embodiment, the one or more than one targeting agent is a plurality of targeting agents, such as for example both amyloid antibody (M31) Fab fragment and an antibody that recognized smooth muscle cells in the cerebrovasculature. In another embodiment, the plurality of targeting agents is two targeting agents. In another embodiment, the plurality of targeting agents is three targeting agents. In another embodiment, the plurality of targeting agents is four targeting agents.

The one or more than one targeting agent can be added to the one or more than one vehicle by any suitable method, as will be understood by those with skill in the art with respect to this disclosure. In one embodiment, the substance comprises an inhibitor, a vehicle and a targeting agent, and making the substance comprises producing the combination of the inhibitor and vehicle, and then adding the targeting agent to the combination by conjugation using a biotin-streptavidin interaction. For example, streptavidin conjugation to amyloid antibody (M31) Fab fragments is accomplished by labeling with the EasyLink Streptavidin Conjugation Kit (Abeam, Cambridge, Mass., US) according to the manufacturer's instructions. Purified Fab fragments are incubated with the modifier and conjugate solution for 3 hours. After quenching the reaction for 30 minutes, the Fab fragments conjugated to avidin are mixed with biotinylated chitosan nanoparticles. Biotinylation of chitosan is accomplished prior to precipitation of chitosan nanoparticles. SulfoNHS-LC-Biotin (Thermo Fisher Scientific Inc., Rockford, Ill., US) is dissolved in PBS and incubated with chitosan flake (0.1% w/v) at room temperature for 3 hours. Free SulfoNHS-LC-Biotin is removed by dialysis. The degree of biotinylation is determined by 2-(4-hydroxyazobenzene) benzoic acid (HABA) assay, with decreases in absorption recorded at 500 nm. However, making the substance comprising the targeting agents can comprise any suitable method, as will be understood by those with skill in the art with respect to this disclosure.

In one embodiment, the substance further comprises one or more than one additional chemical that enables determination of plasmid transfection efficacy where the inhibitor is a plasmid, or enables tracking of the substance. In one embodiment, at least one of the one or more than one additional chemical is selected from the group consisting of hydroxycoumarin and green fluorescent protein. As will be understood by those with skill in the art with respect to this disclosure, hydroxycoumarin and green fluorescent protein can be added to the substance according to techniques known to those with skill in the art, such as adding hydroxycoumarin during precipitation of the chitosan nanoparticles which incorporates the hydroxycoumarin into the structure of the nanoparticles, and chemically modifying green fluorescent protein to link it with streptavidin, which is then introduced to biotinylated nanoparticles formation of the biotinylated nanoparticles.

In some embodiments, the formation of the membrane attack complex of the complement system can be inhibited by a small molecular weight complement inhibitor molecule (“small molecule”) that can be delivered to the targeted site, for example, the cerebrovasculature and/or the cells' adjacent beta-amyloid plaques. The small molecule can be used in addition to, or in place of, the plasmid containing inhibitor or other membrane attack complex inhibitor as described herein. For example, a small molecule that disrupts the formation of the membrane attack complex of the complement system and thereby protects the cells from lysis by the activated complement can be used to inhibit the complement mediated cellular attack. As a result of aging, the brain amyloid cellular clearance is disrupted resulting in the deposition of activated complement and membrane attack complex in the brain and its microvascular. The complement-mediated attack on the microvascular and neuronal cells can contribute to both Alzheimer' s disease and cerebral amyloid angiopathy. A small molecule can be delivered by targeted nanoparticles to specific antigenic determinants in the amyloid deposits of cerebral amyloid angiopathy. Therapeutic benefits can accrue as a result of inhibition of the membrane attack complex to preserve the integrity of both vascular endothelial cells and other neural elements as well as other therapeutic benefits as described herein.

The therapeutic intervention of a targeted delivery of a small molecular weight complement inhibitor molecule that blocks membrane attack complex formation will have a wide range of clinical applications in a variety of autoimmune diseases. In some embodiments, in addition to Alzheimer's disease and cerebral amyloid angiopathy, complement activation is involved in autoimmune diseases including: cryoglobulinemic vasculitis, systemic lupus erythematosus, Sjogren's syndrome, systemic sclerosis, dermatomyositis, and rheumatoid arthritis. The inhibitors and delivery methods disclosed herein can be useful in blocking membrane attack complex formation and thereby preventing cell lysis in these various diseases.

Additionally, in some embodiments, a small molecule that can act as an inhibitor can be delivered via the targeted nanoparticles and/or chitosan microparticles, such as the chitosan nanoparticles described herein. A vehicle for transporting the small molecule to the cerebrovasculature and/or the cells' adjacent beta-amyloid plaques can include similar methods and/or vehicles for transporting as described with reference to the delivery of the plasmid described previously. For example, chitosan nanoparticles, colloidal metallic nanoparticles, polymer nanoparticle, and viral particles can be used to transport the small molecule inhibitor to the target site. In some embodiments, the chitosan delivery vehicle provides a nontoxic, cationic polysaccharide that accretes around negatively charged molecules to nucleate microparticle precipitation as described herein. In some embodiments, the characteristics of the positively charged chitosan surface can enable targeted delivery to the amyloid-laden endothelial surface. In some embodiments, the surface charge and/or proteomic characteristics of the chitosan nanoparticle can be varied to assist in the chitosan nanoparticles ability to be used as a delivery vehicle for various therapeutic techniques.

Chitosan toxicity from endotoxin contamination has previously inhibited full development of this potent therapeutic tool. In some embodiments, chitosan preparations and formulations can allow for detoxification of the known endotoxin contamination of this material. Depyrogenation of the chitosan has been achieved with a nitrogen plasma treatment. Further details of methods and apparatuses of the depyrogenation techniques that are usable with the embodiments described herein are found in the following applications, which are hereby incorporated by reference in their entireties: application Ser. No. 14/097,151, titled “Chitosan-based Hemostatic Textile,” filed Dec. 4, 2013; and U.S. Pat. No. 8,623,274, titled “Chitosan-based Hemostatic Textile,” issued Jan. 7, 2014. Production of purified chitosan can assist in the development of chitosan therapeutics and application to small animals, for example, by allowing for implantable chitosan treatment options.

In some embodiments, the small molecule can include aurin (n)-carboxylic acid or derivatives thereof. Aurin (n)-carboxylic acid disrupts polymerization of C9 and prevents formation of the membrane attack complex of the complement system. The chemical structure of aurin (n)-carboxylic acid is shown below.

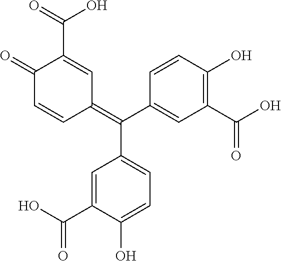 [SMILES: O=C(O)C1=CC(=C(c2ccc(O)c(C(=O)O)c2)c2ccc(O)c(C(=O)O)c2)C=CC1=O]

Aurin (n)-carboxylic acid can be prepared in nitrite-containing sulfuric acid by the condensation of formaldehyde with salicylic acid. Additionally, aurin (n)-carboxylic acid is known to polymerize in aqueous solution and forms a stable free radical. Aurin (n)-carboxylic acid can inhibit protein biosynthesis by inhibiting protein-nucleic acid interactions. In some embodiments, other methods known in the art of preparing and/or polymerizing aurin (n)-carboxylic acid can be utilized to prepare aurin (n)-carboxylic acid for use as an inhibitor. Aurin (n)-carboxylic acid can inhibit formation of the membrane attack complex of the complement system by disrupting the polymerization of C9 and thereby blocking the completed formation of the membrane attack complex.

In some embodiments, the carbonyl, carboxyl, and hydroxyl groups on aurin (n)-carboxylic acid confer a negative charge to the periphery of the molecule, allowing for encapsulation by chitosan. The aurin (n)-carboxylic acid is encapsulated by the appropriately targeted chitosan microparticle as described herein to inhibit the formation of the membrane attack complex of the complement system.

In some embodiments, a substance would be precipitated as previously described, except in place of the plasmid a small molecule would be introduced with a tripolyphosphate (“TPP”) crosslinking agent. Therefore, the substance can comprise the small molecule introduced with the TPP crosslinking agent as the inhibitor instead of the human CD59 cDNA in the pCMV6-AC as the inhibitor as described previously. In some embodiments, the substance comprises the small molecule as the inhibitor introduced with the TPP crosslinking agent, chitosan nanoparticles as the vehicle, and amyloid antibody (M31) Fab or other appropriate anti-amyloid Fab fragments as the targeting agent. The substance binds to beta-amyloid deposition in the cerebrovasculature and releases the small molecule to inhibit the formation of the membrane attack complex by interfering with C8-C9 and/or C9-C9 interactions, thereby disrupting formation of the trans-membrane pore.

In some embodiments, when the small molecule has a negative charge the substitution with the plasmid would be direct and the same procedures described herein for preparation of the substance could be followed. In other embodiments, the small molecule can have a neutral charge. In some embodiments, when the small molecule has a neutral charge, the small molecule could first be encapsulated in cyclodextrin. For example, cyclodextrin (1-5% w/v) can be dissolved in distilled water and 0.01 to 0.5 g of the appropriate small molecule can be added. The solution can be kept under magnetic stirring for 24 hours to allow saturation of cyclodextrin. Precipitation of the substance can proceed as described herein, with the cyclodextrin encapsulation introduced with the TPP crosslinking agent in place of the plasmid.

Chitin, from which chitosan is derived, has been shown to be sufficient to induce clearance of beta-amyloid from the blood vessels of transgenic mice. The ability of induce the clearance of beta-amyloid from the blood vessels allows for the treatment of various diseases as discussed herein. Together with an appropriate inhibitor of the membrane attack complex of the complement system, the therapeutic potential of the treatment modality described herein is very strong, with protection against the membrane attack complex-mediated vessel fragility and concurrent clearance of vascular beta-amyloid. Treatment of an immunotherapy exacerbated cerebral amyloid angiopathy in a transgenic mouse proceeds after in vitro studies and is monitored by serial brain MM determination of BMBs, in vivo optical imaging, and quantitative histopathology.

According to another embodiment, there is provided a pharmaceutical for treating a cerebral amyloid angiopathy related condition or disease. The pharmaceutical comprises one or more than one substance according to the embodiments.

In one embodiment, the pharmaceutical further comprises one or more than one of a binder, a buffer, a coloring chemical, a flavoring chemical and a preservative, as will be understood by those with skill in the art with respect to this disclosure.

According to another embodiment, there is provided a method for treating a cerebral amyloid angiopathy related condition or disease affecting cerebrovasculature in a patient, where the cerebrovasculature comprises tunica intima comprising endothelial cells, tunica media comprising smooth muscle cells, and tunica adventitia, where the condition or disease is associated with an incidence of cytolysis of the smooth muscle cells from beta-amyloid deposition in the cerebrovasculature that leads to formation of membrane attack complex of the complement system in the smooth muscle cells. The method comprises, first, identifying a patient with a cerebral amyloid angiopathy related condition or disease suitable for treatment by the present method. In a preferred embodiment, the patient is a human. In one embodiment, the cerebral amyloid angiopathy related condition or disease is selected from the group consisting of one or more than one of Alzheimer's disease, a brain microbleed, cerebral amyloidosis of the parenchyma, mild cognitive impairment with amyloid plaques in the brain and a combination of the preceding.

In another embodiment, identifying the patient comprises consulting patient records to determine if the patient has a cerebral amyloid angiopathy related condition or disease suitable for treatment by the present method. In another embodiment, identifying the patient comprises diagnosing the patient with a cerebral amyloid angiopathy related condition or disease suitable for treatment by the present method. In one embodiment, diagnosing the patient comprises performing one or more than one of action selected from the group consisting of identifying one or more than one marker for cerebral amyloid angiopathy in blood or another body fluid of the patient, performing cognitive testing, performing an invasive procedure (such as for example biopsying brain tissue of the patient), performing a non-invasive imaging procedure (such as for example computerized tomography, magnetic resonance imaging or ultrasound), and performing a physical examination.

Next, the method comprises providing one or more than one substance that comprises an inhibitor that causes inhibition of the formation of membrane attack complex of the complement system, or comprises providing one or more than one pharmaceutical comprising one or more than one substance that comprises an inhibitor that causes inhibition of the formation of membrane attack complex of the complement system, or comprises providing both one or more than one substance that comprises an inhibitor that causes inhibition of the formation of membrane attack complex of the complement system and one or more than one pharmaceutical comprising one or more than one substance that comprises an inhibitor that causes inhibition of the formation of membrane attack complex of the complement system, where the inhibition by the inhibitor is sufficient to decrease the incidence of or to prevent the incidence of cytolysis of the smooth muscle cells. In one embodiment, one or more than one of the one or more than one substance is a substance according to the embodiments. In another embodiment, one or more than one of the one or more than one pharmaceutical is a pharmaceutical according to the embodiments. In one embodiment, the one or more than one substance is a plurality of substances. In one embodiment, the one or more than one substance is two substances, such as for example chitosan nanoparticles as a vehicle comprising alphaGal lectin as an inhibitor, and chitosan nanoparticles as a vehicle comprising human CD59 cDNA as an inhibitor. In one embodiment, the one or more than one pharmaceutical is a plurality of pharmaceuticals. In one embodiment, the one or more than one pharmaceutical is two pharmaceuticals.

Then, the method comprises administering one or more than one dose of the one or more than one substance or administering one or more than one dose of the one or more than one pharmaceutical to the patient by a route. In one embodiment, the one or more than one dose is one dose. In another embodiment, the one or more than one dose is a plurality of doses. In one embodiment, the plurality of doses is two doses. In another embodiment, the plurality of doses is three doses. In another embodiment, the plurality of doses is four doses. In another embodiment, the plurality of doses is more than four doses. In one embodiment, the one or more than one dose is administered daily for a predetermined amount of time. In another embodiment, the one or more than one dose is administered twice daily for a predetermined amount of time. In another embodiment, the one or more than one dose is administered weekly for a predetermined amount of time. In another embodiment, the one or more than one dose is administered monthly for a predetermined amount of time. In another embodiment, the one or more than one dose is administered between once a day and once a week for a predetermined amount of time. In another embodiment, the one or more than one dose is administered between once a day and once a month for a predetermined amount of time. In one embodiment, the dose is between 0.000001 mg/m² body surface area and 100 g/m² body surface area. In another embodiment, the dose is between 0.0001 mg/m² body surface area and 10 g/m² body surface area. In another embodiment, the dose is between 1 mg/m² body surface area and 1 g/m² body surface area. In one embodiment, the route is selected from the group consisting of intra-arterial injection, intramuscular injection, intranasal spray and intravenous injection.

In one embodiment, the method further comprises determining the effect of treatment on the patient. In one embodiment, determining the effect of treatment on the patient comprises performing one or more than one of action selected from the group consisting of identifying one or more than one marker for cerebral amyloid angiopathy in blood or another body fluid of the patient, performing cognitive testing, performing an invasive procedure (such as for example biopsying brain tissue of the patient), performing a non-invasive imaging procedure (such as for example computerized tomography, magnetic resonance imaging or ultrasound), and performing a physical examination. In another embodiment, the method further comprises adjusting treatment. In one embodiment, adjusting treatment comprising administering to the patient one or more than one additional dose of the one or more than one substance or administering one or more than one additional dose of the one or more than one pharmaceutical to the patient.

### EXAMPLE I

### Determination of the Mechanism of Damage in Cerebral Amyloid Angiopathy

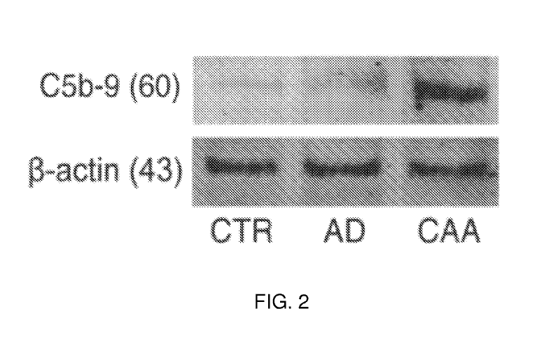

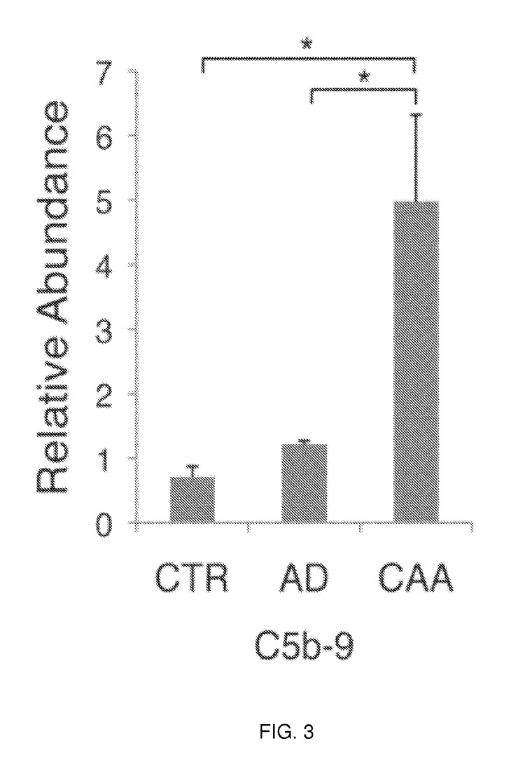

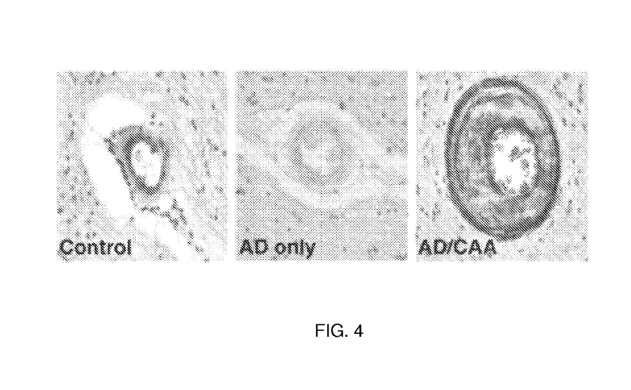

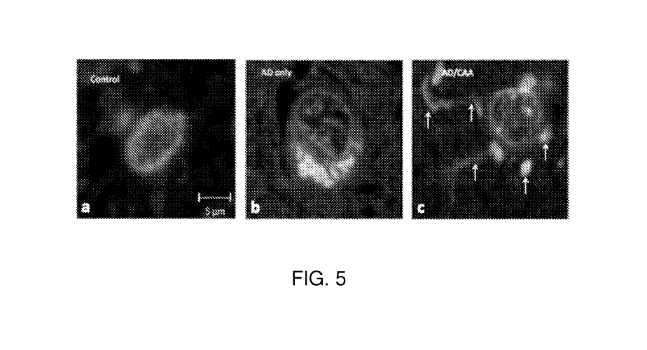

To determine the mechanism of damage in cerebral amyloid angiopathy, a series of studies was performed on blood vessels from frozen postmortem occipital lobe sections that were sonicated on ice, and then probed with antibodies. Referring now to FIG. 2, FIG. 3, FIG. 4 and FIG. 5, there are shown, respectively, western blots showing the accumulation of C6 (upper) and beta-actin (a protein expressed in all cells and used as a loading control to normalize C6 values) (lower) in blood vessel walls from control brain tissue (left), in blood vessel walls from brain tissue from patients diagnosed with Alzheimer's disease (AD, center), and in blood vessel walls from brain tissue from patients diagnosed with Alzheimer's disease concurrent with cerebral amyloid angiopathy (AD/CAA, right) (FIG. 2); a histogram showing the average concentration of C6 from multiple western blots (n=4) in blood vessel walls from control brain tissue, in blood vessel walls from brain tissue from patients diagnosed with Alzheimer's disease (AD), and in blood vessel walls from brain tissue from patients diagnosed with Alzheimer's disease concurrent with cerebral amyloid angiopathy (AD/CAA) (FIG. 3); photomicrographs showing C6 localization in the walls of blood vessels from control brain tissue (left), in the walls of blood vessels from brain tissue from patients diagnosed with Alzheimer's disease (AD, center), and in the walls of blood vessels from brain tissue from patients diagnosed with Alzheimer's disease concurrent with cerebral amyloid angiopathy (AD/CAA, right) by immunohisto-chemistry with DAB-staining (FIG. 4); and confocal micrographs of microglia from control brain tissue (A, left), microglia from brain tissue from patients diagnosed with Alzheimer's disease (B, center), and microglia from brain tissue from patients diagnosed with Alzheimer's disease concurrent with cerebral amyloid angiopathy (C, right), where the brain tissue was stained for alpha2-macroglobulin (a2M) and beta-amyloid (A, B), and for beta-amyloid and CD 11 b (C) (FIG. 5). As can be seen in FIG. 2 and FIG. 3, C6 accumulated more extensively in blood vessel walls of brain tissue from patients diagnosed with Alzheimer's disease concurrent with cerebral amyloid angiopathy than in blood vessel walls of control brain tissue or brain tissue from patients diagnosed with Alzheimer's disease alone. No such pattern was detected for the localization of CD11b. Further, CD11b with beta-amyloid show punctate colocalization at the plasma membrane of microglia from brain tissue from patients diagnosed with Alzheimer's disease concurrent with cerebral amyloid angiopathy as indicated by the arrows (FIG. 5, right), rather than the internalized phagocytic vesicles seen in normal beta-amyloid trafficking of brain tissue from patients diagnosed with Alzheimer's disease alone (FIG. 5, center), where control brain tissue show only slight colocalization for CD11b and beta-amyloid (FIG. 5, left), thereby demonstrating that beta-amyloid is taken up normally via the alpha2-macroglobulin receptor in patients diagnosed with Alzheimer's disease without concurrent cerebral amyloid angiopathy, but is associated with the cell surface receptor CD 11b in the brains of patients diagnosed with Alzheimer's disease concurrent with cerebral amyloid angiopathy. CD11b is known to bind C3b, the molecule at the crossroads of the complement cascade.

These studies demonstrated that the mechanism by which aging microglia in cerebral amyloid angiopathy-damaged brains remove beta-amyloid from cerebral tissues changes from the established alpha2-macroglobulin/LRP-mediated clearance of beta-amyloid (endophagocytosis) to CD11b/C3b receptor-mediated shuttling on the microglial surface (opsonization). The CD11b/C3b receptor/beta-amyloid complex is delivered by microglia to the abluminal vascular wall for disposal of beta-amyloid into the circulatory system. This perpetuates cerebrovascular injury because C3b initiates the complement cascade leading to formation of the membrane attack complex (MAC, C5b-C9), the transmembrane pore part of the innate immune reaction that is the terminal lytic component of the cascade. Membrane attack complex weakens the vessel by destroying cerebrovascular smooth muscle cells (SMC) resulting in vessel fragility, disruption of the blood-brain barrier (BLOOD BRAIN BARRIER) and increasing the probability of brain microbleeds (BMB).

Therefore, these studies identified a treatable pathogenic mechanism for cerebral amyloid angiopathy, namely, inhibiting the formation of membrane attack complex in the cerebrovasculature. In one embodiment, inhibiting the formation of membrane attack complex in the cerebrovasculature, and thus inhibiting induced cytotoxicity in cerebrovascular smooth muscle cells, is accomplished by up regulating levels of CD59 glycoprotein.

### EXAMPLE II

### Method for Treating a Cerebral Amyloid Angiopathy in a Patient

According to one embodiment, a patient with cerebral amyloid angiopathy is treated as follows. First, a patient is identified with Alzheimer's disease associated with cerebral amyloid angiopathy by cognitive analysis followed by brain biopsy. Next, a substance according to the embodiments is provided that comprises human CD59 cDNA in the pCMV6-AC as the inhibitor, chitosan nanoparticles as the vehicle and amyloid antibody (M31) Fab fragments as the targeting agent. Then, the substance is administered to the patient once per week for five weeks at a dose of 1 mg/m2 body surface area, and the once a month thereafter.

The substance binds to the beta-amyloid deposition in the cerebrovasculature. The substance is then endocytosed by cerebrovascular smooth muscle cells and transported by the endosomal/lysosomal pathway to liberate the CD59 cDNA. The CD59 cDNA is processed into mRNA, and the mRNA is transcribed, upregulating CD59 glycoprotein levels. The CD59 blocks the addition of complement component C9 to C5b-8, effectively inhibiting formation of membrane attack complex and decreasing or preventing cytolysis of the smooth muscle cells, and thereby treating the cerebral amyloid angiopathy.

### EXAMPLE III

### Method for Treating a Cerebral Amyloid Angiopathy in a Patient

According to one embodiment, a patient with cerebral amyloid angiopathy is treated as follows. First, a patient is identified with Alzheimer's disease associated with cerebral amyloid angiopathy by cognitive analysis followed by brain biopsy. Next, a substance according to the embodiments is provided. The substance comprises a small molecule that is introduced as the inhibitor with a tripolyphosphate crosslinking agent, chitosan nanoparticles as the vehicle, and amyloid antibody (M31) Fab or other appropriate anti-amyloid Fab fragments as the targeting agent. Then, the substance is administered to the patient once per week for five weeks at a dose of 1 mg/m2 body surface area, and then once a month thereafter.

The substance binds to beta-amyloid deposition in the cerebrovasculature and releases an appropriate small molecule to inhibit the formation of the membrane attack complex by interfering with C8-C9 and/or C9-C9 interactions, thereby disrupting formation of the trans-membrane pore. The inhibition of the formation of membrane attack complex can decrease or prevent cytolysis of the smooth muscle cells, and thereby treating the cerebral amyloid angiopathy.

### EXAMPLE IV

### Impact of Cell Viability Due to the Chitosan-Only Nanparticles

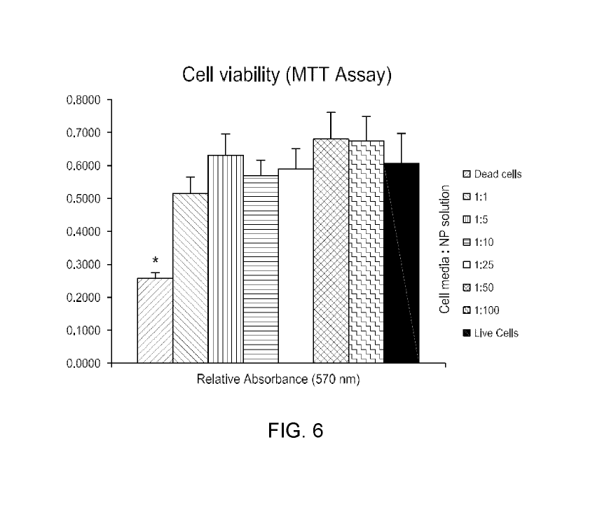

Testing is conducted to determine the impact of cell viability due to the chitosan-only nanoparticles. The nanoparticles tested included nanoparticles of chitosan and a tripolyphosphate (“TPP”) crosslinking agent only. The chart of FIG. 6 illustrates nanoparticle impact on cell viability. Y-axis is absorbance at 495 nm, in arbitrary units.

Colorimetric analysis by 3-(4,5-dimethylthiazol-2-yl)-2,5-diphenyltetrazolium bromide, MTT) of cell viability indicates that treatment with various concentrations of nanoparticles does not harm cells. There is no difference between live control and any treatment group. Dead cell control has diminished signal compared to live control and every treatment group (p<0.05, t-test using SigmaPlot software, n=3). Therefore, the chitosan nanoparticles do not impact cell viability.

While the disclosure has been illustrated and described in detail in the drawings and foregoing description, such illustration and description are to be considered illustrative or exemplary and not restrictive. The disclosure is not limited to the disclosed embodiments. Variations to the disclosed embodiments can be understood and effected by those skilled in the art in practicing the claimed disclosure, from a study of the drawings, the disclosure and the appended claims.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ including but not limited to,' or the like; the term ‘comprising’ as used herein is synonymous with ‘including,’ containing,' or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A substance for treating a cerebral amyloid angiopathy related condition or disease affecting cerebrovasculature in a patient, where the cerebrovasculature comprises tunica intima comprising endothelial cells, tunica media comprising smooth muscle cells, and tunica adventitia, where the condition or disease is associated with an incidence of cytolysis of the smooth muscle cells from beta-amyloid deposition in the cerebrovasculature that leads to formation of membrane attack complex of the complement system in the smooth muscle cells, the substance comprising:
one or more than one inhibitor that causes inhibition of the formation of membrane attack complex of the complement system; and
one or more than one vehicle for transporting the one or more than one inhibitor into the cerebrovasculature;
wherein the inhibition by the inhibitor is sufficient to decrease the incidence of or to prevent the incidence of cytolysis of the smooth muscle cells.

2. The substance of claim 1, where the cerebral amyloid angiopathy related condition or disease is selected from the group consisting of one or more than one of Alzheimer's disease, a brain microbleed, cerebral amyloidosis of the parenchyma, mild cognitive impairment with amyloid plaques in the brain and a combination thereof.

3. The substance of claim 1, where the one or more than one inhibitor specifically causes inhibition of the formation of membrane attack complex of the complement system in the tunica intima of the cerebrovasculature, tunica media of the cerebrovasculature, or both the tunica intima of the cerebrovasculature and tunica media of the cerebrovasculature.

4. The substance of claim 1, where one or more than one of the one or more than one inhibitor that causes inhibition of the formation of membrane attack complex of the complement system upregulates CD59 glycoprotein levels in the cerebrovasculature of the patient.

5. The substance of claim 1, where one or more than one of the one or more than one inhibitor that causes inhibition of the formation of membrane attack complex of the complement system disrupts polymerization of C9.

6. The substance of claim 1, where one or more than one of the one or more than one inhibitor that causes inhibition of the formation of membrane attack complex of the complement system is selected from the group consisting of one or more than one of alphaGal lectin, anti-C5 Mab, C1-Inhibitor, factor H, human CD59 cDNA, a small molecular weight complement inhibitor molecule, and a combination of the preceding.

7. The substance of claim 1, where one of the one or more than one inhibitor that causes inhibition of the formation of membrane attack complex of the complement system is a plasmid comprising human CD59 cDNA.

8. The substance of claim 7, where one of the one or more than one inhibitor comprises human CD59 cDNA in the pCMV6-AC plasmid, or human CD59 cDNA in the pCMV6-XL5 plasmid.

9. The substance of claim 6, where the small molecular weight complement inhibitor molecule is aurin (n)-carboxylic acid or derivatives.

10. The substance of claim 1, where one or more than one of the one or more than one vehicle is selected from the group consisting of chitosan nanoparticles, colloidal metallic nanoparticles, polymer nanoparticles and viral particles.

11. The substance of claim 1, where at least one of the one or more than one vehicle comprises chitosan nanoparticles.

12. The substance of claim 1, further comprising one or more than one targeting agent that recognizes the beta-amyloid deposited in the cerebrovasculature, where one or more than one targeting agent forms at least part of the surface of the substance when one or more than one targeting agent is combined with the inhibitor and the vehicle, thereby directing the substance to the beta-amyloid deposited in the cerebrovasculature when the substance is administered to the patient.

13. The substance of claim 12, where the targeting agent recognizes and attaches to a subset of conformationally unique beta-amyloid deposited in the cerebrovasculature, where the conformationally unique beta-amyloid deposited in the cerebrovasculature is specific for cerebral amyloid angiopathy.

14. The substance of claim 12, where when combined with the inhibitor and the vehicle, the targeting agent directs the substance to the beta-amyloid deposited in the cerebrovascular smooth muscle cells when the substance is administered to the patient.

15. The substance of claim 12, where at least one of the one or more than one targeting agent is a monoclonal antibody or is a fragment of a monoclonal antibody.

16. The substance of claim 12, where at least one of the one or more than one targeting agent is selected from the group consisting of amyloid antibody (M31) Fab fragments, 6E10 beta-amyloid monoclonal antibody and a combination of the preceding.

17. The substance of claim 1, where the substance further comprises one or more than one additional chemical that enables determination of plasmid transfection efficacy where the inhibitor is a plasmid, or enables tracking of the substance.

18. The substance of claim 17, where at least one of the one or more than one additional chemical is selected from the group consisting of hydroxycoumarin and green fluorescent protein.

19. A pharmaceutical for treating a cerebral amyloid angiopathy related condition or disease, the pharmaceutical comprising:
one or more than one substance according to claim 1; and
one or more than one of a binder, a buffer, a coloring chemical, a flavoring chemical and a preservative.

20. A method for treating a cerebral amyloid angiopathy related condition or disease affecting cerebrovasculature in a patient, where the cerebrovasculature comprises tunica intima comprising endothelial cells, tunica media comprising smooth muscle cells, and tunica adventitia, where the condition or disease is associated with an incidence of cytolysis of the smooth muscle cells from beta-amyloid deposition in the cerebrovasculature that leads to formation of membrane attack complex of the complement system in the smooth muscle cells, the method comprising:
identifying a patient with a cerebral amyloid angiopathy related condition or disease suitable for treatment;
providing one or more than one substance that comprises an inhibitor that causes inhibition of the formation of membrane attack complex of the complement system, or comprises providing one or more than one pharmaceutical comprising one or more than one substance that comprises an inhibitor that causes inhibition of the formation of membrane attack complex of the complement system, or comprises providing both one or more than one substance that comprises an inhibitor that causes inhibition of the formation of membrane attack complex of the complement system and one or more than one pharmaceutical comprising one or more than one substance that comprises an inhibitor that causes inhibition of the formation of membrane attack complex of the complement system, where the inhibition by the inhibitor is sufficient to decrease the incidence of or to prevent the incidence of cytolysis of the smooth muscle cells; and
administering one or more than one dose of the one or more than one substance or administering one or more than one dose of the one or more than one pharmaceutical to the patient by a route.
